(19) United States
(12) Patent Application Publication
(10) Pub. No.: US 2008/0255175 A1
(43) Pub. Date: Oct. 16, 2008

(54) Anti-cancer agents, compositions and methods of treating cancers

(76) Inventors: Marina K. Lam, Yardville, NJ (US); Kwok-Leung Shum, East Windsor, NJ (US); Wei Sun, Edison, NJ (US)

Correspondence Address: Alexander R. Pagano, Esq., 440 Speedwell Avenue, Morris Plains, NJ, 07950, US

(21) Appl. No.: 11/787,725

(22) Filed: Apr. 16, 2007

(51) Int. Cl. A61K 31/4745; A61P 35/00; C07D 491/14; C07D 491/147

(52) U.S. Cl. 514/283; 546/48; 546/51

(57) ABSTRACT

Compounds and compositions useful in methods for treating cancer in mammals. The compounds of the invention are of the formula:

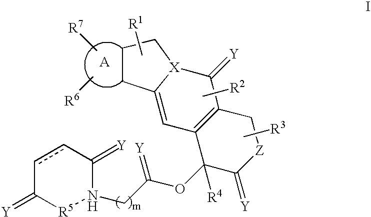

or a pharmaceutically acceptable salt, entantiomer or disastereomer thereof, wherein the “A” ring is a nitrogen-aryl group;
each occurrence of Y is independently oxygen or H₂;
Z is oxygen or —CH₂—;
X is nitrogen or —CH;
R¹, R², R³, R⁴, R⁵, R⁶, and R⁷ are subsitutents;
n is an integer from 1-7;
m is an integer having a value of 1-5; and
the dotted line represents an optional bond.

### FIELD

This invention relates to compounds and compositions for treatment of cancer.

### BACKGROUND

Human topoisomerase I (Topo I) is an enzyme critical to the viability of cellular function that is an attractive target for the design and development of anticancer therapeutics. Eukaryotic DNA Topoisomerase I (Topo I) is an essential nuclear enzyme responsible for the organization and modulation of the topological dilemmas in DNA, such as overwinding, underwinding and catenation. Topo I plays a critical role in allowing a cell to appropriately replicate, transcribe, repair genetic information, and perhaps carry out other DNA processes such as chromatin assembly, recombination and chromosome segregation. Liu, L. F. 58 ANN. REV. BIOCHEM. 351-375 (1989). Schneider, E.; Hsiang, Y-H; Liu, L. F. 21 ADV. PHARMACOL. 149-183 (1990).

Topo I is a 100 kD monomeric protein that catalyzes changes in the topological state of double-stranded DNA (dsDNA) in increments of one linking number. Champoux, J. 29 A J. ADV. PHARMACOL. 71-82 (1994). The three-dimensional structure of human Topo I has been reported. Redinbo, M. R.; Stewart, L.; Kuhn, P.; Champoux, J. J.; Hol, W. G. J. 279 SCIENCE 1504-1513 (1998).

The mechanism by which Topo I acts is believed to proceed through induction of a transient single-stranded break in dsDNA via formation of a covalent protein-DNA adduct referred to as the cleavable complex, so named because these complexes are detected as DNA breaks upon treatment with denaturing agents or alkali. The cleavable complex is formed upon transesterification of a DNA phosphodiester linkage by the active site tyrosine-723 residue on human Topo I, resulting in an ester linkage between the enzyme and the 3′-phosphoryl end of the broken DNA strand. This allows free rotation of the protein-bound 3′ end of the broken DNA strand about the intact complementary DNA strand, resulting in relaxation of the duplex in increments of one linking number. Religation of the broken strand (via a second transesterification reaction) and subsequent dissociation of topoisomerase I completes the catalytic cycle.

Topoisomerase I poisons act via stabilization of the cleavable complex, mediated by formation of a ternary complex consisting of drug, topoisomerase I and DNA. Pommier, Y.; Tanizawa, A.; Kohn, K. W. 29B ADV. PHARMACOL. 73-92 (1994). Agents such as camptothecin (the prototype topoisomerase I poison) do not bind to DNA directly, nor to topoisomerase I alone, but only to topoisomerase I complexed with DNA. It has been postulated that the stabilized DNA-protein-drug complex causes lethal DNA strand breaks upon collision with the advancing replication fork. It is by this mechanism that the topoisomerase I poison converts the enzyme into a DNA damaging agent, resulting in disruption of DNA replication and, eventually, cell death. This postulate is supported by the fact that camptothecin is highly phase-specific, only killing cells in S-phase.

It has been reported that intracellular levels of topo I are elevated in a number of human solid tumors, relative to the respective normal tissues, suggesting that variations in topo I levels are tumor type specific. Giovanella, B. P. et al. 246 SCIENCE 1046-1048 (1989); Lima, C. D. Wang, J. C.; Mondragon, A. 367 NATURE 138-146 (!994); Husain, I et al. 54 CANCER RES. 539-546 (1994). Thus, topo I represents a promising target for the development of new cancer chemotherapeutic agents against a number of solid tumors. Development of anti-topo I agents offers a new approach to the multi-regimental arsenal of therapies currently used in the clinic for the treatment of cancer.

Camptothecin is a water-insoluble, cytotoxic alkaloid produced by Camptotheca accuminata trees indigenous to China and Nothapodytes foetida trees indigenous to India. Wall et al., 88 J. AMER. CHEM. SOC. 3888 (1966). Camptothecins belong to the topoisomerase inhibitor class of antineoplastic agents, specifically inhibiting the action of the nuclear enzyme topoisomerase I which is involved in DNA replication. Hsiang, et al., 48 CANCER RES. 1722-1726 (1988). Parameters for antitumor activity in the camptothecin family have been established. Wall et al, 17 ANN. REV., PHARMACOL. TOXICOL. 117, (1977). These results indicate that an intact lactone ring and the a-hydroxyl group are essential for antitumor activity. Id. The structure of camptothecin is shown below. Heckendorf et al., 41 J. ORG. CHEM. 2045 (1976).

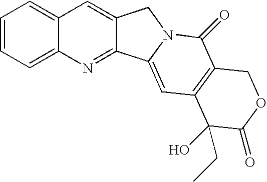

Camptothecin was of interest at the time of its initial isolation due to its noteworthy activity in the mouse leukemia L 1210 system. 23 CHEM. REV. 385 (1973); 60 CANCER TREAT. REP. 1007 (1967). Earlier data for the antitumor activity of camptothecin were obtained by employing experimentally transplanted malignancies such as leukemia L 1210 in mice, or Walker 256 tumor in rats. Id.

Topotecan (Hycamtin™) is an analog of camptothecin approved by the US Food and Drug Administration (FDA) for the treatment of certain types of cancer.

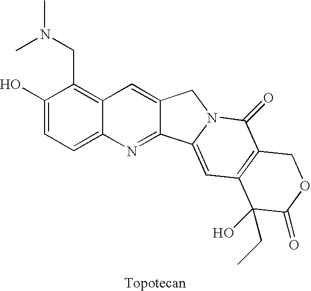

Topotecan, exhibits a cell cycle-specific mechanism of action, acting during S-phase (DNA replication) to cause irreversible double strand breaks in DNA that ultimately lead to G2 cell cycle arrest and apoptosis.

In the free-base form, topotecan has a broad spectrum of activity against a range of tumor cell lines and murine allograft and human xenograft tumor models. See, e.g., McCabe, F. L. et al, CANCER INVEST 12:308 313 (1994); Emerson, et al, 55 CANCER RES. 603-609 (1995); Thompson, 1400 BIOCHIM. BIOPHYS. ACTA 301-319 (1998); Ormrod, et al., 58 DRUGS 533-551 (1999); Hardman, et al., 19 ANTICANCER RES. 2269-2274 (1999).

More recently, evidence has emerged that topotecan has strong anti-angiogenic properties that may contribute to its anti-tumor mechanism of action. O'Leary, et al., 5 CLIN. CANCER RES. 181-187 (1999); Clements, et al., 44 CANCER CHEMOTHER. PHARMACOL. 411 416 (1999). All these treatments are associated with dose-limiting toxicity such as non-cumulative myelosuppression leading to anaemia, neutropenia and thrombocytopenia, and gastrointestinal-related toxicity, including mucositis and diarrhea. Clinically, topotecan has been approved for second-line therapy in ovarian and small cell lung cancer (SCLC) and is currently the focus of extensive clinical evaluation.

A number of attempts have been made to provide other derivatives of camptothecin having greater biological activity and enhanced stability. Many of these compounds are the products of modifications on the rings of the molecule, but few of these modifications have enhanced the stability of the lactone ring under physiological conditions. A need still exists for new camptothecin -derivatives and associated delivering systems for medical purposes.

### SUMMARY

The invention relates to compounds and compositions useful in methods for treating or preventing cancer in mammals.

Inn one embodiment, the invention relates to compounds of the formula:

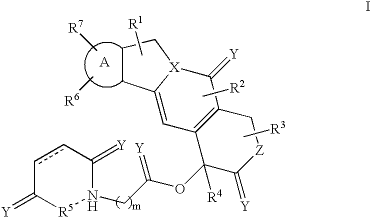

or a pharmaceutically acceptable salt, entantiomer or disastereomer thereof, wherein the “A” ring is a nitrogen-aryl group;
each occurrence of Y is independently oxygen or H₂;
Z is oxygen or —CH₂—;
X is nitrogen or —CH;

R¹, R², R³, R⁴, R⁵, R⁶, and R⁷ are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—(C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C₁-C₈)alkynyl, S-aryl; O—S(O)₂—(C₁-C₈)alkyl, O—S(O)₂—C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer from 1-7;
m is an integer having a value of 1-5; and
the dotted line represents an optional bond.
   In another embodiment, the invention relates to the formula:

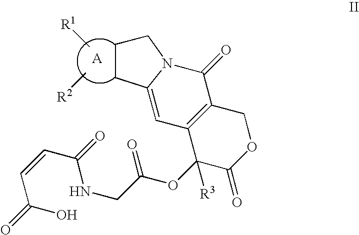

or a pharmaceutically acceptable salt, entantiomer or disastereomer thereof, wherein,
the “A” ring is a nitrogen-aryl group;

R¹ and R² are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—(C₁-C₈)alkyl, O—C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C₁-C₈)alkynyl, S-aryl; O—S(O)₂—(C₁-C₈)alkyl, O—S(O)₂—(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer from 1-7; and
R³ is H, (C₁-C₈)alkyl, (C₁-C₈)alken1yl, or (C₁-C₈)alkynyl.
   In still another embodiment, the invention relates to the of the formula:

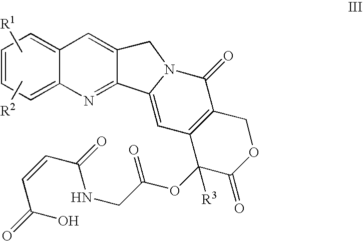

or a pharmaceutically acceptable salt, entantiomer or disastereomer thereof, wherein

R¹ and R² are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—(C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C₁-C₈)alkynyl, or S-aryl; O—S(O)₂—(C₁-C₈)alkyl, O—S(O)₂—(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer having a value of 1 to 7; and
R³ is H, (C₁-C₈)alkyl, (C₁-C₈)alkenyl, or (C₁-C₈)alkynyl.

Alternate embodiments of the invention relate to methods of treating cancer in a mammal comprising administering to a mammal in need of said treatment or prevention a therapeutically effective amount of a compound of the formulas I, II or III listed above.

In another embodiment of the invention the compounds of the invention inhibit

In another embodiment, the invention relates to methods for modulating cellular function in a mammal comprising administering an modulatorily effective amount of a compound of the formulas I, II or III listed above or an enantiomer, stereoisomer or diastereoisomer thereof to the mammal.

In another embodiment, the invention relates to methods of modulating cellular function of a mammal suffering from a disease or medical condition comprising locally administering a therapeutically effective amount of a compound of the formulas I, II or III listed above or an enantiomer, stereoisomer or diastereoisomer thereof to a mammal suffering from said disease or condition, whereby the disease or medical condition is treated.

In another embodiment, the invention relates to the use of compounds of the formulas I, II or III listed above or an enantiomer, stereoisomer or diastereoisomer thereof, or a pharmaceutically acceptable salt thereof, in the manufacture of a medicament for the treatment or prevention of cancer.

In another aspect, the invention relates to pharmaceutical formulations for modulating cellular function in a mammal comprising a therapeutically effective amount of a compound of the formulas I, II or III listed above or an enantiomer, stereoisomer or diastereoisomer thereof in a pharmaceutically acceptable carrier.

### DETAILED DESCRIPTION

### 1. COMPOUNDS OF THE INVENTION

The invention relates to compound of the formula:

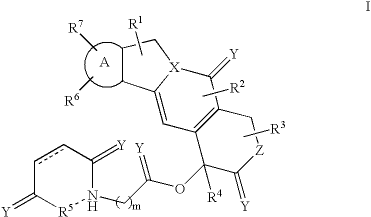

or a pharmaceutically acceptable salt, entantiomer or disastereomer thereof, wherein the “A” ring is a nitrogen-aryl group;
each occurrence of Y is independently oxygen or H₂;
Z is oxygen or —CH₂—;
X is nitrogen or —CH;

R¹, R², R³, R⁴, R⁵, R⁶, and R⁷ are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—C₁-C₈)alkyl, S—(C₁-C₈)alkenyl, S—(C₁-C₈)alkynyl, S-aryl; O—S(O)₂—(C₁-C₈)alkyl, O—S(O)₂—(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer from 1-7;
m is an integer having a value of 1-5; and
the dotted line represents an optional bond.

The compounds of the invention and compositions thereof are useful to treat cancer. The compounds of the invention are prophylactically effective and can therefore be used to prevent cancer. Cancers that can be treated or prevented according to the invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adeno carcinomas, cystadeno carcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

Individual compounds of the invention include, but are not limited to the compounds listed in Table 1 below.

TABLE 1
COMPOUNDS OF THE INVENTION

| Compound No. | Compound Structure |
|---|---|
| 1 | 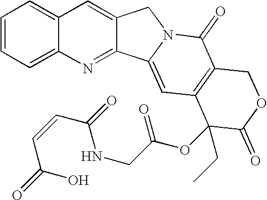 |
| 2 | 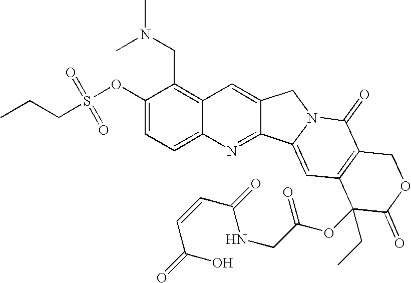 |
| 3 | 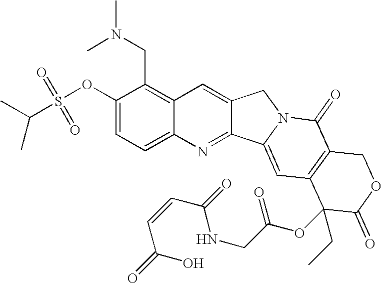 |

### 2. ISOMERIC PURITY AND ISOLATION

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass the racemic form of compounds of the invention as well as all enantiomers and stereoisomers, that is, both the stereomerically pure form (e.g., geometrically pure, enantiomerically pure, or diastereomerically pure) and enantiomeric and stereoisomeric mixtures.

A compound of the invention is considered optically active or enantiomerically pure (i.e., substantially the R-form or substantially the S-form) with respect to a chiral center when the compound is about 90% ee (enantiomeric excess) or greater, preferably, equal to or greater than 95% ee with respect to a particular chiral center. A compound of the invention is considered to be in enantiomerically enriched form when the compound has an enantiomeric excess of greater than about 80% ee, preferably greater than about. As used herein, a racemic mixture means about 50% of one enantiomer and about 50% of is corresponding enantiomer relative to all chiral centers in the molecule. Thus, the invention encompasses all enantiomerically pure, enantiomerically enriched, and racemic mixtures of compounds of the invention.

Enantiomeric and stereoisomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well known asymmetric synthetic methods.

When administered to a patient, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, the compounds of the invention are purified by conventional techniques. As used herein, “purified” means that when isolated, the isolate contains at least 95%, preferably at least 98%, of a single compound of the invention (or an enantiomeric or diastereomeric mixture thereof) by weight of the isolate.

### 3. DEFINITIONS

### 3.1 “Treat” “Treating” and “Treatment”

The terms “treat,” “treating” and “treatment,” as used herein, contemplate an action that occurs while a patient is suffering from the specified disease or disorder, which reduces the severity of the disease or disorder.

### 3.2 “Therapeutically Effective Amount”

As used herein, the term “therapeutically effective amount” means the amount of a compound of the invention that will elicit a biological or medical response in the mammal that is being that is being treated by a medical doctor or other clinician.

### 3.3 “Prophylactically Effective”, “Preventing” or “Preventive”

As used herein, the term “prophylactically effective” or “preventive” means the amount of a compound of the invention that will prevent or inhibit affliction a medical condition that a medical doctor or other clinician is trying to prevent, inhibit before a patient begins to suffer from the specified disease or disorder.

### 3.4 “Pharmaceutically Acceptable Salt(s)”

The term “pharmaceutically acceptable salt(s)”, as used herein includes but is not limited to salts of acidic or basic groups that may be present in the compounds of the invention. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds of the invention that include an amino moiety also can form pharmaceutically acceptable salts with various amino acids, in addition to the acids mentioned above. Compounds of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

### 3.5 “Alkyl Group”

As used herein, the term “alkyl group” means a saturated, monovalent, unbranched or branched hydrocarbon chain. Examples of alkyl groups include, but are not limited to, (C₁-C₆)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or substituted with one or two suitable substituents.

### 3.6 “Alkenyl Group”

As used herein, the term “alkenyl group” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂-C₆)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

### 3.7 “Alkynyl Group”

As used herein, the term “alkynyl group” means monovalent, unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted with one or two suitable substituents.

### 3.8 “Aryl Group”

As used herein, the term “aryl group” means a monocyclic or polycyclic-aromatic radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or two suitable substituents. Preferably, the aryl group is a monocyclic ring, wherein the ring comprises 6 carbon atoms, referred to herein as “(C₆)aryl”.

### 3.9 “Heteroaryl Group”

As used herein, the term “heteroaryl group” means a monocyclic- or polycyclic aromatic ring comprising carbon atoms, hydrogen atoms, and one or more heteroatoms, preferably 1 to 3 heteroatoms, independently selected from nitrogen, oxygen, and sulfur. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, furyl, phienyl, isoxazolyl, and oxazolyl. A heteroaryl group can be unsubstituted or substituted with one or two suitable substituents.

In one embodiment, a heteroaryl group is a monocyclic ring, wherein the ring comprises 2 to 5 carbon atoms and 1 to 3 heteroatoms, referred to herein as “(C₂-C₅)heteroaryl”.

The meaning of the term heteroaryl group encompasses “nitrogen-aryl group” as that term is defined below.

### 3.10 “Nitrogen-aryl Group”

As used herein, the term “nitrogen-aryl group” means a polycyclic aromatic ring, preferably a bicyclic or tricyclic ring, the ring comprising carbon atoms, hydrogen atoms, and one or more nitrogen atoms, preferably 1, 2, 3 or 4 nitrogen atoms, preferably 1 or 2 nitrogen atoms, more preferably 1 nitrogen atom. Illustrative examples of nitrogen-aryl groups include, but are not limited to, quinolinyl, isoquinolinyl, benzo[g]quinolinyl, benzo[g]isoquinolinyl, acridinyl, cinnolinyl, quinazolinyl, 1,8-naphthyridinyl, 1,7-naphthyridinyl, 1,6-naphthyridinyl, 1,5-naphthyridinyl, quinoxalinyl, phthalazinyl, benzo[g]phthalazinyl, benzo[g]quinazolinyl, benzo[b][1,6]naphthyridinyl, pyrido[3,4-g]quinoline, pyrido[3,4-g]isoquinoline, pyrido[4,3-g]isoquinolinyl, pyrido[4,3-g]quinolinyl, benzo[b][1,7]naphthyridinyl, benzo[g]cinnolinyl, benzo[g]cinnolinyl, benzo[b][1,7]naphthyridinyl, pyrido[4,3-g]quinolinyl, pyrido[4,3-g]isoquinolinyl, pyrido[3,4-g]isoquinolinyl, pyrido[3,4-g]quinolinyl, benzo[b][1,6]naphthyridinyl, benzo[g]quinazolinyl, pyrido[3,2-g]quinazolinyl, and pyrido[3,4-b][1,5]naphthyridine. A nitrogen-aryl group can be unsubstituted or substituted with one or two suitable substituents.

### 3.11 “Cycloalkyl Group”

As used herein, the term “cycloalkyl group” means a monocyclic or polycyclic saturated ring comprising carbon and hydrogen atoms and having no carbon--carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted by one or two suitable substituents. Preferably, the cycloalkyl group is a monocyclic ring or a bicyclic ring.

### 3.12 “Heterocycloalkyl Group”

As used herein, the term “heterocycloalkyl group” means a monocyclic or polycyclic ring comprising carbon and hydrogen atoms and at least one heteroatom, preferably, I to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as (C₁-C₆)heterocycloalkyl.

### 3.13 ““Heterocyclic Radical” or “Heterocyclic Ring””

As used herein, the terms “heterocyclic radical” or “heterocyclic ring” mean a heterocycloalkyl group or a heteroaryl group.

### 3.14 “Alkoxy Group”

As used herein, the term “alkoxy group” means an —O-alkyl group, wherein alkyl is as defined above. An alkoxy group can be unsubstituted or substituted with one or two suitable substituents. Preferably, the alkyl chain of an alkyloxy group is from I to 6 carbon atoms in length, referred to herein as “(C₁-C₆)alkoxy”.

### 3.15 “Aryloxy Group”

As used herein, the term “aryloxy group” means an —O-aryl group, wherein aryl is as defined above. An aryloxy group can be unsubstituted or substituted with one or two suitable substituents. Preferably, the aryl ring of an aryloxy group is a monocyclic ring, wherein the ring comprises 6 carbon atoms, referred to herein as “(C₆)aryloxy

### 3.16 “Benzyl”

As used herein, the term “benzyl” means —CH₂-phenyl.

### 3.17 “Phenyl”

As used herein, the term “phenyl” means —C₆H₅. A phenyl group can be unsubstituted or substituted with one or two suitable substituents.

### 3.18 “Carbonyl”

As used herein, a “carbonyl” group is a divalent group of the formula —C(O)—.

### 3.19 “Alkoxycarbonyl”

As used herein, the term “alkoxycarbonyl” group means a monovalent group of the formula —C(O)-alkoxy. Preferably, the hydrocarbon chain of an alkoxycarbonyl group is from 1 to 8 carbon atoms in length, referred to herein as a “lower alkoxycarbonyl” group

### 3.20 “Carbamoyl”

As used herein, the term “carbamoyl” group means the radical —C(O)N(R′)₂, wherein R′ is chosen from the group consisting of hydrogen, alkyl, and aryl.

### 3.21 “Halogen”

As used herein, the term “halogen” means fluorine, chlorine, bromine, or iodine. Correspondingly, the meaning of the terms “halo” and “Hal” encompass fluoro, chloro, bromo, and iodo.

### 3.22 “Suitable Substituent”

As used herein, the term “suitable substituent” means a group that does not nullify the synthetic, therapeutic or pharmaceutical utility of the compounds of the invention or the intermediates useful for preparing them. Examples of suitable substituents include, but are not limited to: (C₁-C8)alkyl; (C₁-C₈)alkenyl; (C₁-C₈)alkynyl; aryl; (C₂-C₅)heteroaryl; (C₁-C₆)heterocycloalkyl; (C₃-C₇)cycloalkyl; O—(C₁-C₈)alkyl; O—(C₁-C₈)alkenyl; O—(C₁-C₈)alkynyl; O-aryl; CN; OH; oxo; halo; C(O)OH; COhalo; O(CO)halo; CF₃; N₃; NO₂; NH₂; NH((C₁-C₈)alkyl); N((C₁-C₈)alkyl)₂; NH(aryl); N(aryl)₂; (CO)NH₂; (CO)NH((C₁-C₈)alkyl); (CO)N((C₁-C₈)alkyl)₂; (CO)NH(aryl); (CO)N(aryl)₂; O(CO)NH₂; NHOH; NOH((C₁-C₈)alkyl); NOH(aryl); O(CO)NH((C₁-C₈)alkyl); O(CO)N((C₁-C₈)alkyl)₂; O(CO)NH(aryl); O(CO)N(aryl)₂; CHO; CO((C₁-C₈)alkyl); CO(aryl); C(O)O((C₁-C₈)alkyl); C(O)O(aryl); O(CO)((C₁-C₈)alkyl); O(CO)(aryl); O(CO)O((C₁-C₈)alkyl); O(CO)O(aryl); S—(C₁-C₈)alkyl; S—(C₁-C₈)alkenyl; S—(C₁-C_{C}₈)alkynyl; and S-aryl. One of skill in art can readily choose a suitable substituent based on the stability and pharmacological and synthetic activity of the compound of the invention.

### 4. SYNTHESIS OF COMPOUNDS OF THE INVENTION

The compounds of the invention can be obtained via standard, synthetic methodology. Some convenient methods are illustrated in Schemes 1-3. Starting materials useful for preparing the compounds of the invention and intermediates therefor, are commercially available or can be prepared from commercially available materials using known synthetic methods and reagents.

Protecting groups utilized herein denote groups which generally are not found in the final therapeutic compounds but which are intentionally introduced at some stage of the synthesis in order to protect groups which otherwise might be altered in the course of chemical manipulations. Such protecting groups are removed or converted to the desired group at a later stage of the synthesis and compounds bearing such protecting groups thus are of importance primarily as chemical intermediates (although some derivatives also exhibit biological activity). Accordingly, the precise structure of the protecting group is not critical. Numerous reactions for the formation and removal of such protecting groups are described in a number of standard works including, for example, “Protective Groups in Organic Chemistry”, Plenum Press, London and New York, 1973; Greene, Th. W. “Protective Groups in Organic Synthesis”, Wiley, N.Y., 1981; “The Peptides”, Vol. I, Schroder and Lubke, Academic Press, London and New York, 1965; “Methoden der organischen Chemie”, Houben-Weyl, 4th Edition, Vol.15/I, Georg Thieme Verlag, Stuttgart 1974, the disclosures of which are incorporated herein by reference.

Scheme 1 below illustrates general methodology for the general synthesis of compounds of the invention of the formula I.

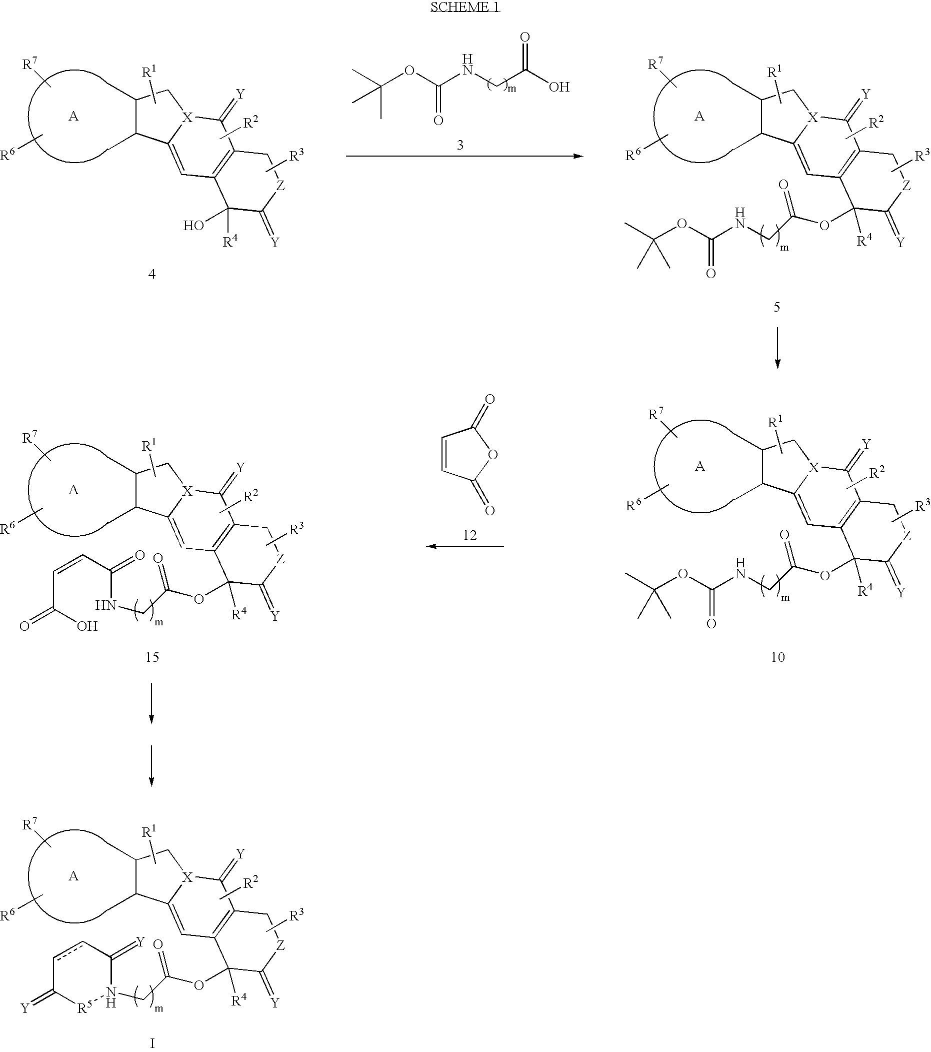

General synthesis of compounds 5: Starting compounds 4 are prepared by methods well known in the art, or are commercially available. A solution comprising 1 (3.5 mmol), and a Lewis acid, such as scandium triflate (2 mmol), and a amine base, such as dimethylaminopyridine (10 mmol) in an anhydrous organic solvent, such as methylene chloride (80 mL to 100 mL) is cooled, for example, to −8° C. in an ice-salt bath, for about 0.5 hours followed by addition of 2-(tert-butoxycarbonylamino)acetic acid (10 mmol) and the reaction mixture is stirred at −8° C. for about half hour. A coupling agent is then added, such a dicyclohexylcarbodiimide (17 mmol) and the mixture is stirred at −8° C. for an additional 0.5 h before allowing to warm to room temperature. The room-temperature mixture is stirred for 24 hours and filtered. The filtrate washed with 0.1 N HCl, 30 mL, distilled water, and 30 mL saturated NaCl solution. The organic phase is dried, for example, using anhydrous MgSO₄, filtered and, concentrated. Compound 5 is then purified, for example, by recrystallization using standard methods.

General synthesis of compounds 10: Trifluoroacetic acid (6 mL) is added to a solution of 5 (1.6 g) in an organic solvent, such as methylene chloride (20 mL), and stirred for 0.5 h. The mixture is evaporated to small volume, a non-polar organic solvent, such as toluene is added, and further evaporation removes residual trifluoroacetic acid. Compound 10 is purified by standard methods in the art, such as recrystallization from methanol.

General synthesis of compounds I: A solution of compound 10 (3.5 mmol), an organic base, such picoline (17 mmol), maleic anhydride (20 mmol) and a polar organic solvent such as dimethylformamide (25 mL) is stirred in an ice bath about 8 hrs. The solvent is evaporated and the product is purified by standard methods in the art, for example, by silica gel column chromatography. Further treatment of the product, for example, couplings, cyclizations, reductions, oxidations and/or hydrogenation according to well known methods provides compounds of formula I.

In an alternate embodiment, compounds of formula II are prepared according to Scheme 2 below.

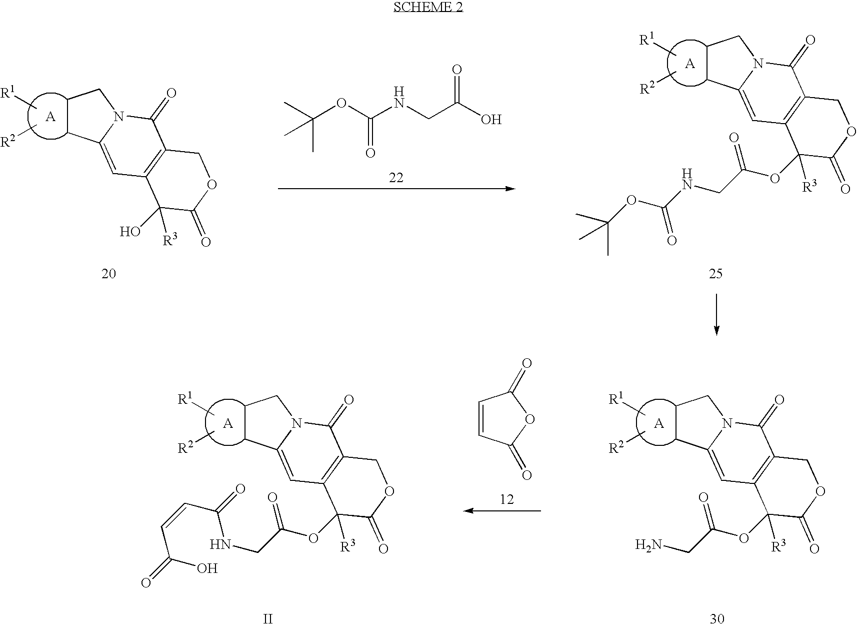

General synthesis of compounds 25: A solution of starting compound 20 (0.8 mmol) commercially available or prepared by well known methods, a Lewis acid, such as scandium triflate (0.5 mmol), and an amine base, such as dimethylaminopyridine (2.25 mmol) in an anhydrous organic solvent, such as methylene chloride (60 mL) is cooled, for example, to −8° C. in an ice-salt bath, for half an hour followed by addition of 2-(tert-butoxycarbonylamino)acetic acid (4 mmol) and the reaction mixture is stirred at −8° C. for half hour, then a coupling agent, such as dicyclohexylcarbodiimide (5 mmol) is added and stirring continued at −8° C. for another 0.5 hours. The mixture is allowed to warm to room temperature and stirred during a 24 hour period. The reaction mixture is filtered and the filtrate evaporated to dryness. Compound 25 is purified and isolated by standard methods in the art, such as recrystallization from methanol-ether.

Synthesis of compounds 30: trifluoroacetic acid (4 mL) is added to a solution of 25 (0.5 g) in an organic solvent, such as methylene chloride (20 mL), and stirred for 0.5 h. The mixture is evaporated and a non-polar solvent, such as toluene, is added and further evaporation removes residual trifluoroacetic acid. The product is purified by well known methods, for example, ether can be added to give solid precipitate, then filtration yields a solid, which can be further purified for example, by recrystallization from methanol.

General synthesis of compounds II: A solution of 30 (0.8 mmol), an organic base, such as picoline (0.28 g, 0.23 mmol), maleic anhydride (0.25 mmol) in a polar organic solvent, such as dimethylformamide (20 mL), is cooled, for example, in ice bath and stirred at for about 6 hours. The solvent is evaporated and the product is purified by standard methods in the art, for example, by silica gel column chromatography

In an alternate embodiment, compounds of formula III are prepared according to Scheme 3 below.

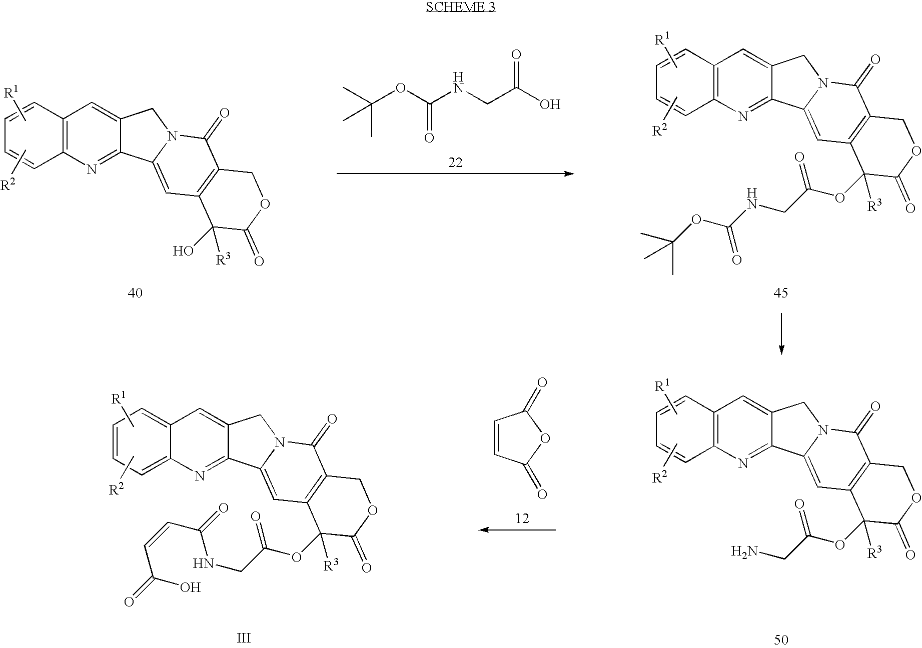

General synthesis of compounds 45: A solution of starting compound 40 (0.8 mmol) commercially available or prepared by well known methods, a Lewis acid, such as scandium triflate (0.5 mmol), and an amine base, such as dimethylaminopyridine (2.3 mmol) in an anhydrous organic solvent, such as methylene chloride (60 mL) is cooled, for example, to −8° C. in an ice-salt bath, for 0.5 hours followed by addition of 2-(tert-butoxycarbonylamino)acetic acid (4 mmol). The reaction mixture is stirred at −8° C. for 0.5 hours, then a coupling agent, such as dicyclohexylcarbodiimide (5 mmol), is added and stirring is continued at −8° C. for another 0.5 hours. The mixture is allowed to warm to room temperature and stirred during a 24 hour period. The reaction mixture is filtered and the filtrate evaporated to dryness. Compound 45 is purified and isolated by standard methods in the art, such as recrystallization from methanol-ether.

Synthesis of compounds 50: trifluoroacetic acid (4 mL) is added to a solution of 45 (0.48 g) in an organic solvent, such as methylene chloride (20 mL), and stirred for 0.5 h. The mixture is evaporated and a non-polar solvent, such as toluene is added and further evaporation removes residual trifluoroacetic acid. The product is purified by well known methods, for example, ether can be added to give solid, then filtration yields a solid, which can be further purified, for example, by recrystallization from methanol.

General synthesis of compounds III: A solution of 50 (0.8 mmol), an organic base, such as picoline (0.28 g, 0.23 mmol), and maleic anhydride (0.25 mmol) in a polar organic solvent, such as dimethylformamide (20 mL) is cooled, for example, in ice bath at 0° C. and stirred at for about 6 hrs. The solvent is evaporated and the product is purified by standard methods in the art, for example, by silica gel column chromatography.

### 5. ADMINISTRATION AND THERAPEUTIC APPLICATION

The present invention provides methods for the treatment or prevention of cancer, comprising administering to a patient a therapeutically effective amount of a compound of the invention or a composition comprising a compound of the invention and a pharmaceutically acceptable vehicle, excipient, or diluent. Cancers that can be treated or prevented by administering the compounds or the compositions of the invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adeno carcinomas, cystadeno carcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

In one embodiment, the compounds and compositions of the invention are administered orally or parenterally. The compounds and compositions of the invention may also be administered by any other convenient route, for example, by intravenous infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one compound of the invention is administered to a patient.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds of the invention into the bloodstream.

In specific embodiments, it may be desirable to administer one or more compounds of the invention locally to the area in need of treatment or prevention. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site of a tumor or malignacy.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

The present compounds and compositions will contain a therapeutically effective amount of a compound of the invention, optionally more than one compound of the invention, preferably in purified form, together with a suitable amount of a pharmaceutically acceptable vehicle, excipient, or diluent so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and compositions of the invention and pharmaceutically acceptable vehicle, excipient, or diluents are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compounds and compositions, if desired, can also contain minor amounts of wetting or emulsifying agents, or pH buffering agents.

The present compounds and compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule. Other examples of suitable pharmaceutical vehicles are described in “Remington's Pharmaceutical Sciences” by E. W. Martin.

In a preferred embodiment, the compounds and compositions of the invention are formulated in accordance with routine procedures as a pharmaceutical composition adapted for intravenous administration to human beings. Typically, compounds and compositions of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by intravenous infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compounds and compositions of the invention for oral delivery may be in the form of tablets, lozenges, aqueous or oily suspensions, granules, powders, emulsions, capsules, syrups, or elixirs. Compounds and compositions of the invention for oral delivery can also be formulated in foods and food mixes. Moreover, where in tablet or pill form, the compounds and compositions may be coated to delay disintegration and absorption in the gastrointestinal tract thereby providing a sustained action over an extended period of time. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a compound of the invention that will be effective in the treatment or prevention of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compounds and compositions will also depend on the route of administration, and the seriousness of the disease or disorder, and should be decided according to the judgment of the practitioner and each patient's circumstances.

By way of example, suitable dosage ranges for oral administration in humans are generally about 0.01 milligrams to about 100 milligrams of a compound of the invention per kilogram body weight. In specific preferred embodiments of the invention, the oral dose is about 0.05 milligrams to about 50 milligrams per kilogram body weight, more preferably, about 0.1 milligrams to about 25 milligrams per kilogram body weight, more preferably about 0.5 milligrams to about 15 milligrams per kilogram body weight, and yet more preferably about 1 milligrams to about 10 milligrams per kilogram body weight. In a most preferred embodiment, the oral dose is 5 milligrams of a compound of the invention per kilogram body weight.

The dosage amounts described herein refer to total amounts administered; that is, if more than one compound of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 milligram to 50 milligrams per kilogram body weight, 0.05 milligram to 25 milligrams per kilogram body weight, and 0.1 milligram to 10 milligrams per kilogram body weight. Effective doses may be extrapolated from dose-response curves derived from in vitro or animal model test systems. Such animal models and systems are well known in the art.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred to treat. The compounds and compositions of the invention may also be demonstrated to be effective and safe using animal model systems.

### 5.1 Combination Therapy

In certain embodiments, a compound of the invention is administered to a mammal, preferably, a human concurrently with one or more other therapeutic agents, or with one or more other compounds of the invention, or with both. By “concurrently” it is meant that a compound of the invention and the other agent are administered to a mammal in a sequence and within a time interval such that the compound of the invention can act together with the other agent or agents to provide an increased or synergistic benefit than if they were administered otherwise. For example, each component may be administered at the same time or sequentially in any order at different points in time; however, if not administered at the same time, they should be administered sufficiently closely in time so as to provide the desired treatment or prevention effect. Preferably, all components are administered at the same time, and if not administered at the same time, preferably, they are all administered from about 6 hours to about 12 hours apart from one another.

When used in combination with other therapeutic agents, such as other anti-cancer compounds, the compounds of the invention and the therapeutic agent can act additively or, more preferably, synergistically. In one embodiment, a compound or a composition of the invention is administered concurrently with another therapeutic agent in the same pharmaceutical composition. In another embodiment, a compound or a composition of the invention is administered concurrently with another therapeutic agent in separate pharmaceutical compositions. In still another embodiment, a compound or a composition of the invention is administered prior or subsequent to administration of another therapeutic agent. As many of the disorders for which the compounds and compositions of the invention are useful in treating are chronic disorders, in one embodiment combination therapy involves alternating between administering a compound or a composition of the invention and a pharmaceutical composition comprising another therapeutic agent, e.g., to minimize the toxicity associated with a particular drug. In certain embodiments, when a composition of the invention is administered concurrently with another therapeutic agent or agents that potentially produce adverse side effects including, but not limited to toxicity, the therapeutic agent can advantageously be administered at a dose that falls below the threshold that the adverse side effect is elicited.

Examples of anti-cancer drugs that can be used in combination with compounds of the invention include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; dacarbazine; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

### 6. EXAMPLES

### 6.1 Example 1

### Synthesis of Compound 1

Compound 1 was synthesized according to the Scheme below.

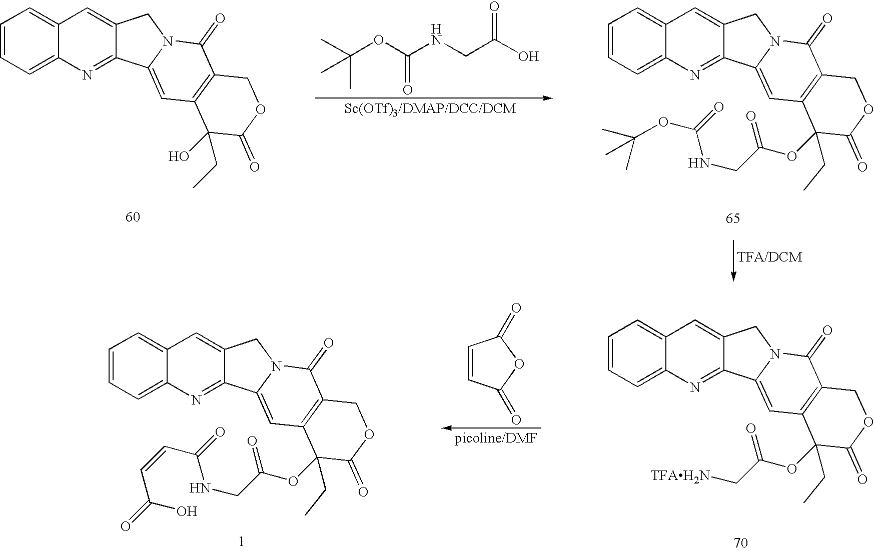

A solution of camptothecin (60, 1.2 g, 3.45 mmol), scandium triflate (1.018 g, 2.07 mmol), and dimethylaminopyridine (1.264 g, 10.3 mmol) in anhydrous methylene chloride (80 mL) was cooled to −8° C. in an ice-salt bath for0.5 hours followed by addition of 2-(tert-butoxycarbonylamino)acetic acid (1.8 g, 10.3 mmol) and the reaction mixture was stirred at −8° C. for half hour, then added dicyclohexylcarbodiimide (3.557 g, 17.2 mmol) and continued stirring at −8° C. for another half a hour before it was warmed up to room temperature for 24 hours. The reaction mixture was filtered and the filtrate was washed by 2×30 mL 0.1 N HCl, 30 mL distilled water, and 30 mL saturated NaCl solution. The organic phase was dried over anhydrous MgSO₄, filtered and, evaporated to dryness. Recrystallization from methanol-ether gave the product 65 (1.58 g, 91%). The structure of 65 was confirmed by ¹H and ¹³C NMR.

Compound 70. A solution of 65 (1.58 g) in methylene chloride (20 mL) was added trifluoroacetic acid (6 mL) and stirred for 0.5 h, the color of the solution changed from light yellow to dark green during the stirring. The mixture was evaporated to small volume, toluene was added and further evaporation removed residue of trifluoroacetic acid. Ether was then added to give solid, filtration gave brownish yellow solid, which was recrystallized from methanol to give yellow product 70 (1.973 g). The structure of 70 was confirmed by ¹H and ¹³C NMR.

Compound 1. A solution of 70 (1.793 g, 3.45 mmol), picoline (1.606 g, 17.2 mmol), and maleic anhydride (1.014 g, 20.7 mmol) in DMF (25 mL) in ice bath at 0° C. was stirred at for 8 hrs. The solvent was evaporated and the product was purified by silica gel column chromatograph to give pure IV (0.85 g, 49%). The structure of IV was confirmed by ¹H and ¹³C NMR. Melting point: 250-252° C.

### 6.2 Example 2

### Synthesis of Compound 2

Compound 2 was synthesized according to the Scheme below.

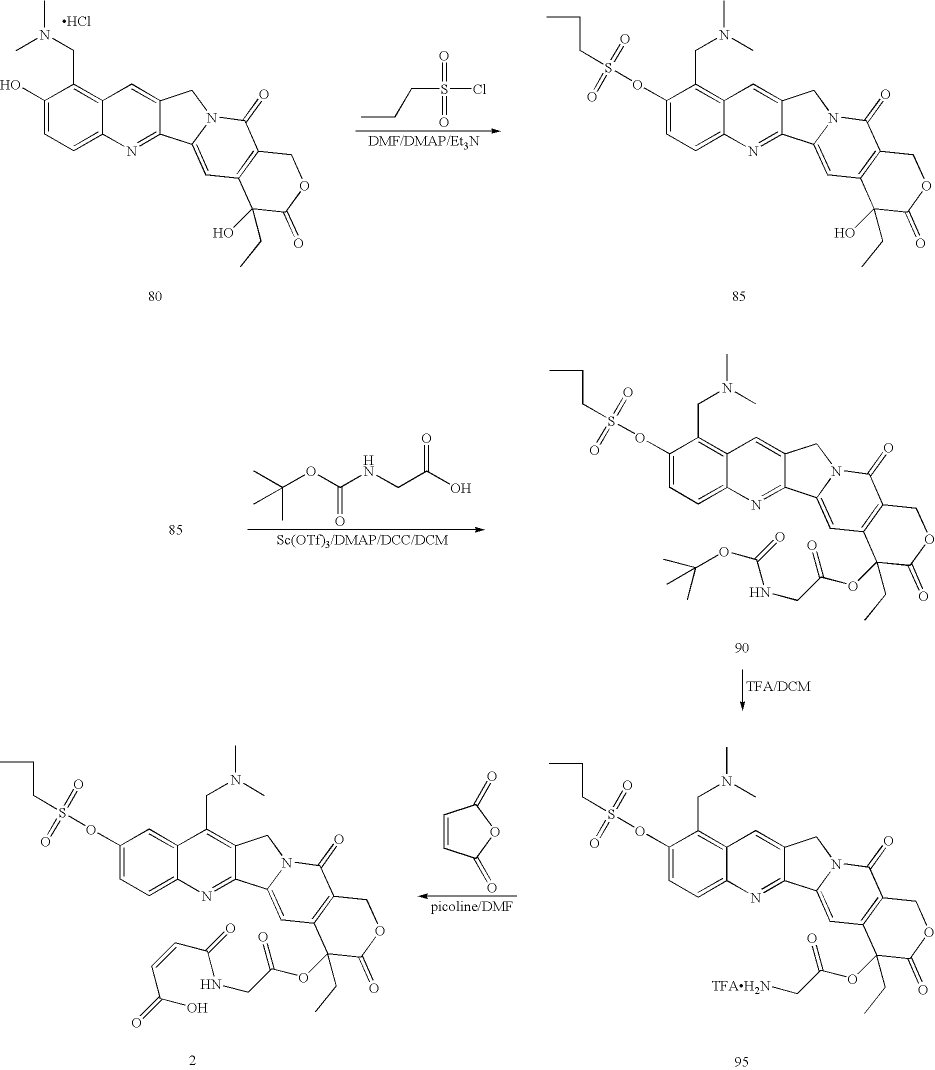

Compound 85. A solution of Topotecan 80 (0.20 g, 0.44 mmol), dimethylaminopyridine (0.04 g, 0.33 mmol) in DMF (10 mL) at 0° C. was added triethylamine (0.62 mL, 4.40 mmol). It was then added a 5 mL solution of 1-propylsulfonic chloride (0.40 mL, 3.52 mmol) dropwise and stirred at 0° C. for 6 hours. Water (20 mL) was added to the reaction mixture, and extracted by methylene chloride (20 mL×3). The pH of the aqueous layer was adjusted to 7 with 10% HCl. The solid precipitated which was filtered to give light yellow solid 85 (0.11 g, 55%). The structure of 85 was confirmed by ¹H and ¹³C NMR. Melting point: 94-96° C.

Compound 90. A solution of 85 (0.4 g, 0.76 mmol), scandium triflate (0.224 g, 0.456 mmol), and dimethylaminopyridine (0.278 g, 2.28 mmol) in anhydrous methylene chloride (60 mL) was cooled to −8° C. in an ice-salt bath for half an hour followed by addition of 2-(tert-butoxycarbonylamino)acetic acid (0.664 g, 3.80 mmol) and the reaction mixture was stirred at −8° C. for half hour, then added dicyclohexylcarbodiimide (1.1 g, 5.3 mmol) and continued stirring at −8° C. for another half a hour before it was warmed up to room temperature for 24 hours. The reaction mixture was filtered and the filtrate evaporated to dryness. Recrystallization from methanol-ether gave the product 90 (0.48 g, 92.2%). The structure of 90 was confirmed by ¹H and ¹³C NMR.

Compound 95. A solution of 90 (0.48 g) in methylene chloride (20 mL) was added trifluoroacetic acid (4 mL) and stirred for 0.5 h, the color of the solution changed from yellow to dark yellow during the stirring. The mixture was evaporated to small volume, toluene was added and further evaporation removed residue of trifluoroacetic acid. Ether was then added to give solid, filtration gave brownish yellow solid, which was recrystallized from methanol to give yellow product 90 (0.55 g). The structure of 90 was confirmed by ¹H and ¹³C NMR.

Compound 2. A solution of 90 (0.53 g, 0.76 mmol), picoline (0.28 g, 0.23 mmol), and maleic anhydride (0.224 g, 0.228 mmol) in DMF (20 mL) in ice bath at 0° C. was stirred at for 6 hrs. The solvent was evaporated and the product was purified by silica gel column chromatograph to give pure 2 (0.26 g, 50%). The structure of 2 was confirmed by ¹H and ¹³C NMR (spectra attached). Melting point: 195-196° C.

### 6.3 Example 3

### Synthesis of Compound 3

Compound 3 was synthesized according to the Scheme below.

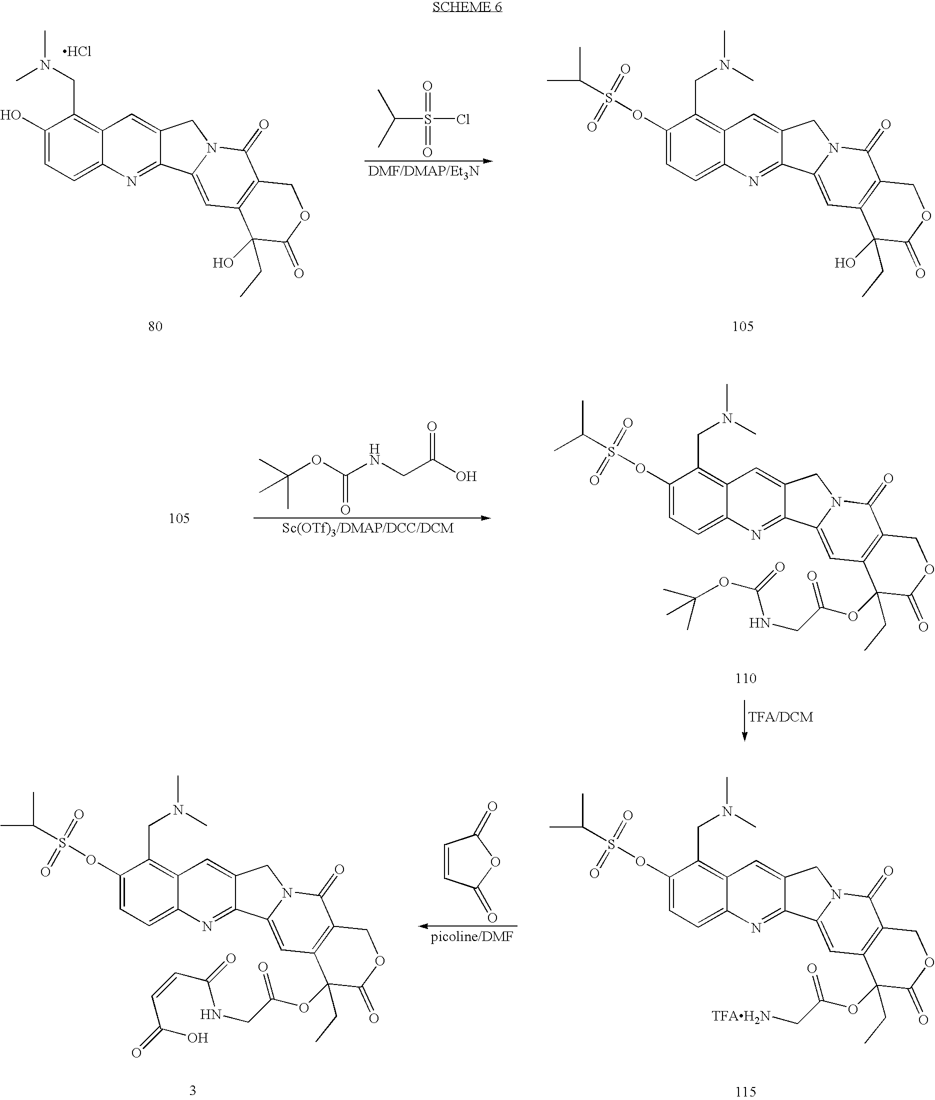

Compound 105. A solution of Topotecan 80 (0.20 g, 0.44 mmol), dimethylaminopyridine (0.04 g, 0.33 mmol) in DMF (10 mL) at 0° C. was added triethylamine (0.92 mL, 3.30 mmol). It was then added a 5 mL solution of 2-propylsulfonic chloride (0.76 mL, 6.60 mmol) dropwise and stirred at 0° C. for 5 hours. Water (30 mL) was added to the reaction mixture, and extracted by methylene chloride (20 mL×3). The pH of the aqueous layer was adjusted to 7 with 10% HCl. The solid precipitated which was filtered to give light yellow solid 105 (0.14 g, 70%). The structure of 105 was confirmed by ¹H and ¹³C NMR. Melting point: 142-144° C.

Compound 110. A solution of 105 (0.2 g, 0.38 mmol), scandium triflate (0.112 g, 0.23 mmol), and dimethylaminopyridine (0.139 g, 1.14 mmol) in anhydrous methylene chloride (30 mL) was cooled to −8° C. in an ice-salt bath for half an hour followed by addition of 2-(tert-butoxycarbonylamino)acetic acid (0.332 g, 1.90 mmol) and the reaction mixture was stirred at −8° C. for half hour, then added dicyclohexylcarbodiimide (0.548 g, 2.65 mmol) and continued stirring at −8° C. for another half a hour before it was warmed up to room temperature for 24 hours. The reaction mixture was filtered and the filtrate evaporated to dryness. Recrystallization from methanol-ether gave the product 110 (0.25 g, 96.1%). The structure of 110 was confirmed by ¹H and ¹³C NMR.

Compound 115. A solution of 110 (0.25 g) in methylene chloride (10 mL) was added trifluoroacetic acid (2 mL) and stirred for 0.5 h, the color of the solution changed from yellow to dark green during the stirring. The mixture was evaporated to small volume, toluene was added and further evaporation removed residue of trifluoroacetic acid. Ether was then added to give solid, filtration gave brownish yellow solid, which was recrystallized from methanol to give yellow product 115 (0.264 g). The structure of 115 was confirmed by ¹H and ¹³C NMR.

Compound 3. A solution of 115 (0.264 g, 0.38 mmol), picoline (0.141 g, 0.152 mmol), and maleic anhydride (0.112 g, 0.114 mmol) in DMF (10 mL) in ice bath at 0° C. was stirred at for 6 hrs. The solvent was evaporated and the product was purified by silica gel column chromatograph to give pure 3 (0.11 g, 42%). The structure of 3 was confirmed by ¹H and ¹³C NMR (spectra attached). Melting point: 173-175° C.

### 6.4 Example 4

### Evaluation The Anticancer Activity of Compounds of the Invention 1, 2 and 3 on Nude Mice Xenografted NCI-H460 Human Non-Small Cell Lung Cancer According to the SFDA Anticancer New Drug Preclinical R&D Guidelines

In this study the NCI-H460 human non-small cell lung cancer nude mice xenograft model was established and applied for in vivo anti-cancer activity evaluation of three compounds of the invention.

BALB/c nude mice, were provided by Shanghai SLAC Experimental Animal Co., Ltd. (Experimental animal production license:SCXK (Shanghai) 2003-0003; Experimental animal application license:SYXK (Jiangsu) 2002-0053). The ages of the mice was 35-40 days; their body weight was 18-22 g; half of the mice were female and the other half were male. Eight nude mice were used per group. The compounds used in this study are listed in Table 2 below.

TABLE 2
COMPOUNDS OF THE INVENTION TESTED IN THIS STUDY

| Compound | | | | |
|---|---|---|---|---|
| No. | Structure | Drug Prep | Dosage 1 | Dosage 2 |
| 1 | 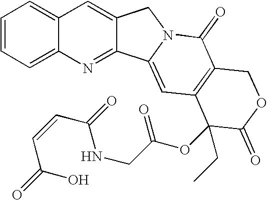 | 1 mg/ml | 0.625 mg/kg | 0.3125 mg/kg |
| 2 | 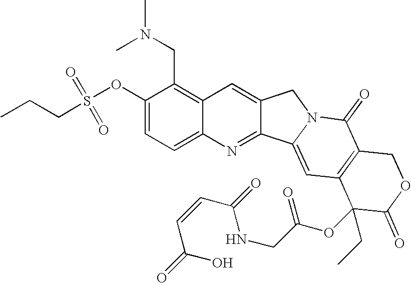 | 2 mg/ml | 5 mg/kg | 2.5 mg/kg |
| 3 | 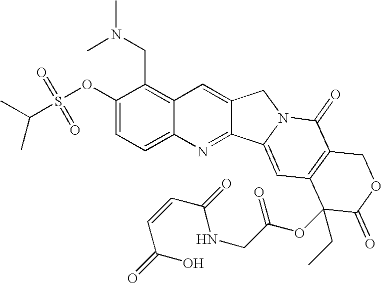 | 1 mg/ml | 1.5625 mg/kg | 0.78125 mg/kg |
| Control | 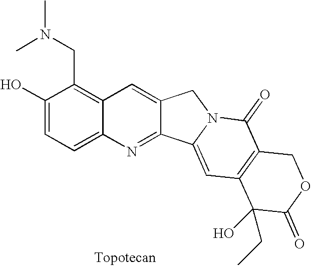 | 5.7 mg/ml | 2 mg/kg | |

The dosage of the three compounds listed in Table 2 above was designed according to the result of the mouse acute toxicity performance. The dosage in nude mice of the three compounds was the highest safety dosage in acute toxicity at which no obvious side effects were observed.

NCI-H460 human non-small cell lung cancer nude mice xenograft model was established by s.c. injection of 1×106 NCI-H460 cells into nude mice axilla. After formation of tumor the xenograft was passaged for three generations in nude mice.

The tumor growing in Log phase was isolated from nude mice and sliced into 1.5 mm³ particles. Under SPF conditions, the tumor particles were inoculated s.c. of nude mice right axilla. The xenografted tumor was measured the length and width by vernier caliper. Nude mice were randomly divided into eight groups when the bearing tumor reached 100-300mm³ volume. Tumor length and width were measured twice every week to dynamic evaluate the anticancer activity of the tested compounds. Compound 1 was given by i.p. twice every week, compound 2 was given by i.v. twice every week, compound 3 was given by i.v. twice every week, control Topotecan was given by i.v. twice every week. In model group saline was give i.v. twice every week. After four weeks drug administration, nude mice were sacrificed and tumor was isolated by surgery for weight detection.

Tumor volume (TV) was calculated by the following formula: TV=1/2×a×b2 a and b represent length and width, respectively.

The anticancer effect of the three compounds of the invention on NCI-H460 human lung cancer xenograft model is shown in Tables 3 and 4. The results indicate that compounds of the invention 1, 2 and 3 inhibit nude mice xenograft NCI-H460 human non-small cell lung cancer growth. The inhibitory rate of compound 3 given i.v. at the dosage of 1.5625mg/kg and 0.78125 mg/kg was 55.8% and 46.4% , respectively. The inhibitory rate of compound 2 given i.v. at the dosage of 5mg/kg and 2.5 mg/kg was 49.7% and 43.2%, respectively. The inhibitory rate of compound 1 given i.p. at the dosage of 0.625mg/kg and 0.3125 mg/kg was 42.5% and 39.9% respectively. The inhibitory rate of topotecan given i.v. at the dosage of 2mg/kg was 48.7%.

TABLE 3
Growth Inhibitory Effect of Compounds of the invention on NCI - H460
Human Non-Small Cell Lung Cancer Nude Mice Xenograft (X ± SD, n = 8)

| | | | | | Tumor |
|---|---|---|---|---|---|
| | | Starting Body | Ending Body Weight | Tumor Weight | Inhibitory |
| Groups | Dosage | Weight (g) | (g) | (g) | Growth Weight |
| Model | — | 18.34 ± 1.78 | 22.07 ± 1.68 | 3.08 ± 0.88 | — |
| topotecan | 2 | 17.91 ± 1.34 | 17.32 ± 1.18** | 1.58 ± 0.74** | 48.7 |
| Compound 1 | 0.625 | 18.14 ± 1.83 | 18.86 ± 1.60** | 1.77 ± 0.73** | 42.5 |
| Compound 1 | 0.3125 | 18.54 ± 1.24 | 18.59 ± 1.46** | 1.85 ± 0.75** | 39.9 |
| Compound 2 | 5 | 17.74 ± 1.56 | 18.93 ± 1.49** | 1.55 ± 0.51** | 49.7 |
| Compound 2 | 2.5 | 18.11 ± 1.47 | 17.82 ± 1.06** | 1.75 ± 0.90** | 43.2 |
| Compound 3 | 1.5625 | 17.96 ± 1.71 | 19.63 ± 0.77** | 1.36 ± 0.50** | 55.8 |
| Compound 3 | 0.78125 | 18.18 ± 1.34 | 18.42 ± 1.04** | 1.65 ± 0.86** | 46.4 |

TABLE 4
Effect of Compounds of the Invention on Tumor Volume Change Of NCI - H460 Human Lung Cancer Xenografted
In Nude Mice (X ± SD, n = 8, mm3)

| | Dosage | | | | | | | | |
|---|---|---|---|---|---|---|---|---|---|
| Group | (mg/kg) | 0.5 week | 1 week | 1.5 weeks | 2 weeks | 2.5 weeks | 3 weeks | 3.5 weeks | 4 weeks |
| Model | — | 223.0 ± | 635.9 ± | 1269.8 ± 524.3 | 1525.3 ± 592.3 | 1606.2 ± 712.5 | 1994.7 ± 795.8 | 2222.7 ± 1109.2 | 2440.6 ± |
| | | 117.2 | 205.5 | | | | | | 1023.3 |
| topotecan | 2 | 209.6 ± | 574.0 ± | 759.8 ± 419.9* | 933.1 ± 496.8* | 1220.7 ± 611.4 | 1273.3 ± 685.1 | 1413.0 ± 717.0 | 1639.9 ± |
| | | 65.9 | 387.8 | | | | | | 888.2 |
| Compound 1 | 0.625 | 220.9 ± | 504.2 ± | 706.7 ± 450.3* | 1104.7 ± 759.6 | 1367.1 ± 880.2 | 1566.7 ± 867.8 | 1798.2 ± 767.2 | 2088.1 ± |
| | | 178.6 | 338.2 | | | | | | 950.3 |
| Compound 1 | 0.3125 | 194.9 ± | 560.8 ± | 766.0 ± 480.3 | 1245.7 ± 585.6 | 1459.9 ± 793.3 | 1638.9 ± 827.1 | 1909.7 ± 464.4 | 2209.8 ± |
| | | 84.7 | 383.8 | | | | | | 760.6 |
| Compound 2 | 5 | 205.2 ± | 413.8 ± | 602.1 ± 339.3** | 733.7 ± 497.0* | 950.0 ± 578.0 | 1147.2 ± 598.0* | 1632.9 ± 752.6 | 1639.4 ± |
| | | 115.8 | 296.6 | | | | | | 687.3 |
| Compound 2 | 2.5 | 182.6 ± | 472.2 ± | 706.8 ± 360.0* | 861.1 ± 523.9* | 1084.9 ± 536.3 | 1269.1 ± 644.1 | 1841.8 ± 859.5 | 2029.4 ± |
| | | 74.4 | 327.9 | | | | | | 766.3 |
| Compound 3 | 1.5625 | 237.9 ± | 392.9 ± | 547.2 ± 390.0** | 719.6 ± 315.8** | 958.4 ± 486.2 | 1093.2 ± 692.2* | 1400.4 ± 670.6 | 1588.3 ± |
| | | 185.2 | 287.8 | | | | | | 867.9 |
| Compound 3 | 0.78125 | 211.8 ± | 455.2 ± | 624.6 ± 371.5* | 795.4 ± 389.0* | 999.8 ± 562.9 | 1291.5 ± 564.4 | 1553.2 ± 706.3 | 1908.5 ± |
| | | 155.2 | 259.8 | | | | | | 639.5 |

In conclusion, the anticancer activity of three compounds of the invention on NCI-H460 human non-small cell lung cancer was Compound 3> Compound 2> Compound 1. The compound 1, 2 and 3 showed dose-dependent anticancer effect. All the three compounds showed inhibitory effect on mice body weight gain. The result showed that compounds of the invention 1, 2 and 3 could inhibit nude mice xenograft NCI-H460 human non-small cell lung cancer growth. The inhibitory rate of compound 3 given i.v. at the dosage of 1.5625 mg/kg and 0.78125 mg/kg was 55.8% and 46.4% , respectively. The inhibitory rate of compound 2 given i.v. at the dosage of 5mg/kg and 2.5 mg/kg was 49.7% and 43.2% , respectively. The inhibitory rate of compound 1 given i.v. at the dosage of 0.625 mg/kg and 0.3125 mg/kg was 42.5% and 39.9%, respectively. The inhibitory rate of Topotecan (as a control) given i.v. at the dosage of 2 mg/kg was 48.7%. In conclusion, the anticancer activity of three compounds of the invention on NCI-H460 human non-small cell lung cancer was Compound 3> Compound 2> Compound 1.

### 6.5 Example 5

### In vitro Efficacy: The Compounds were tested in a Cell Proliferation 96-welled in vitro MTT assay

The tests were done in triplicate and the test samples included negative control, positive control and ACB compounds at several concentrations (10⁻⁴ to 10⁻⁸) in 0.4% DMSO/DW medium. The incubation time was 72 hr and the IC₅₀ was calculated from the generated inhibition curves. The summary of efficacy data of three ACB compounds and positive control of topotecan are listed in the following table.

### In Vitro MTT Assay IC50 Activity

| Cancer | | | | |
|---|---|---|---|---|
| Strains & | ACB-1 | ACB-2 | ACB-3 | Topotecan |
| Types | (μM) | (μM) | (μM) | (μM) |
| HT-29, | 44.3 | 50.8 | 14.3 | 22.9 |
| Human | | | | |
| Colorectal | | | | |
| Lewis Lung, | 6.34 | 12.8 | 0.929 | 11.0 |
| mouse lung | | | | |
| P388, Mouse | 10.9 | 10.6 | 0.0847 | 18.5 |
| Lymphoma | | | | |
| MCF-7, | 20.4 | 30.8 | 9.79 | 22.2 |
| Human | | | | |
| Breast | | | | |

Other methods will be known to the skilled artisan and are within the scope of the invention. The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

What is claimed is:

1. A compound of the formula: 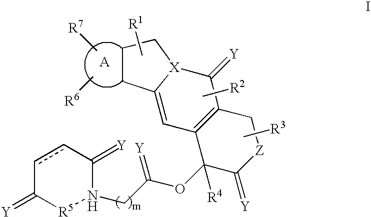
or a pharmaceutically acceptable salt, entantiomer or disastereomer thereof, wherein the “A” ring is a nitrogen-aryl group;
each occurrence of Y is independently oxygen or H₂;
Z is oxygen or —CH₂—;
X is nitrogen or —CH;
R¹, R², R³, R⁴, R⁵, R⁶, and R⁷ are independently H, (C₁-C₈)alkyl, (C₁-C₈)alkenyl, C₈)alkynyl, aryl, (C₂-C₅)heteroaryl, (C₁-C₆)heterocycloalkyl, (C₃-C₇)cycloalkyl, O—(C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C₁-C₈)alkynyl, S-aryl; O—S(O)₂—(C₁-C₈)alkyl, O—S(O)₂—(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer from 1-7;
m is an integer having a value of 1-5; and
the dotted line represents an optional bond.

2. The compound of claim 1 wherein:
R⁶ is H or a group of the formula: 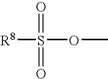
wherein R⁸ is (C₁-C₈)alkyl, (C₁-C₈)alkenyl, (C₁-C₈)alkynyl, or aryl; and
R⁷ is H or a group of the formula: 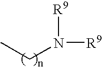
wherein each occurrence of R⁹ is independently H, (C₁-C₈)alkyl, (C₁-C₈)alkenyl, (C₁-C₈)alkynyl, or aryl, and n is an integer having a value of 0-7;

3. The compound of claim 2 wherein R³ and R⁸, and R⁹ are independent (C₁-C₈)alkyl groups.

4. The compound of claim 1 wherein R¹, R², R³, R⁴, R⁵ are all H.

5. The compound of claim 1 wherein the “A” ring is of a formula:

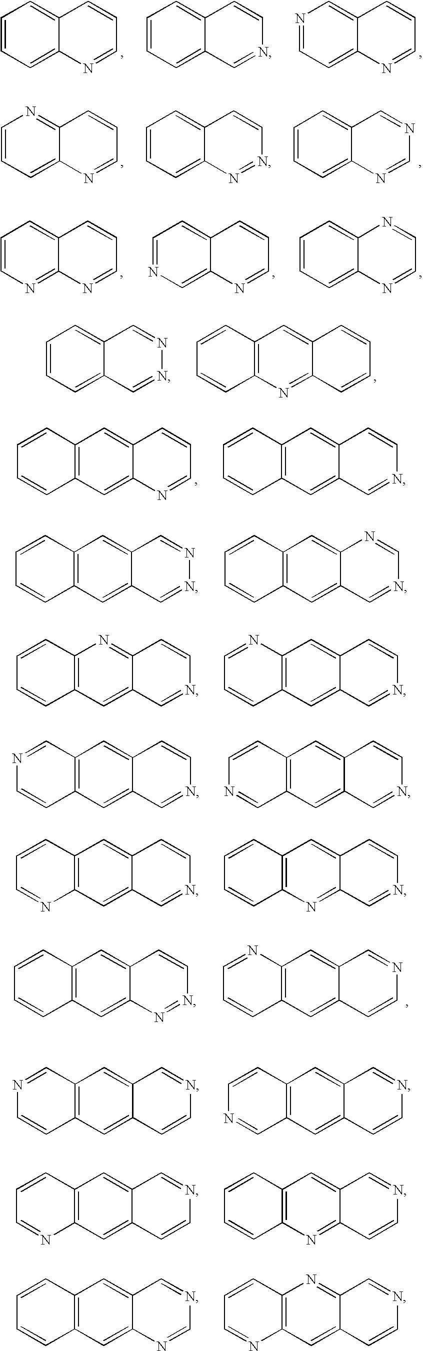

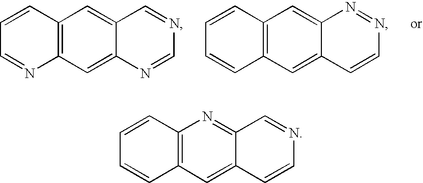

6. The compound of claim 1, wherein each occurrence of Y is oxygen.

7. The compound of claim 1 wherein Z is oxygen.

8. The compound of claim 1 wherein R⁴ is (C₁-C₈)alkyl.

9. The compound of claim 1 further comprising a pharmaceutically acceptable carrier, excipient, or diluent.

10. A compound of the formula: 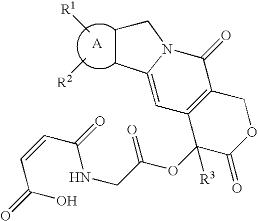
or a pharmaceutically acceptable salt, entantiomer or disastereomer thereof, wherein, the “A” ring is a nitrogen-aryl group;
R¹ and R² are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—(C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C₁-C₈)alkynyl, S-aryl; O—S(O)₂—C₁-C₈)alkyl, O—S(O)₂—(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer from 1-7; and
R³ is H, (C₁-C₈)alkyl, (C₁-C₈)alkenyl, or (C₁-C₈)alkynyl.

11. The compound of claim 10 wherein R¹ is H or a group of the formula: 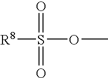
wherein R⁸ is (C₁-C₈)alkyl, (C₁-C₈)alkenyl, (C₁-C₈)alkynyl, or aryl; and
R² is H or a group of the formula: 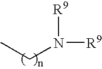
wherein each occurrence of R⁹ is independently H, (C₁-C₈)alkyl, (C₁-C₈)alkenyl, (C₁-C₈)alkynyl, or aryl, and n is an integer having a value of 0-7.

12. The compound of claim 11 wherein R³ and R⁸, and R⁹ are independent (C₁-C₈)alkyl groups.

13. The compound of claim 10 wherein the “A” ring is of the formula:

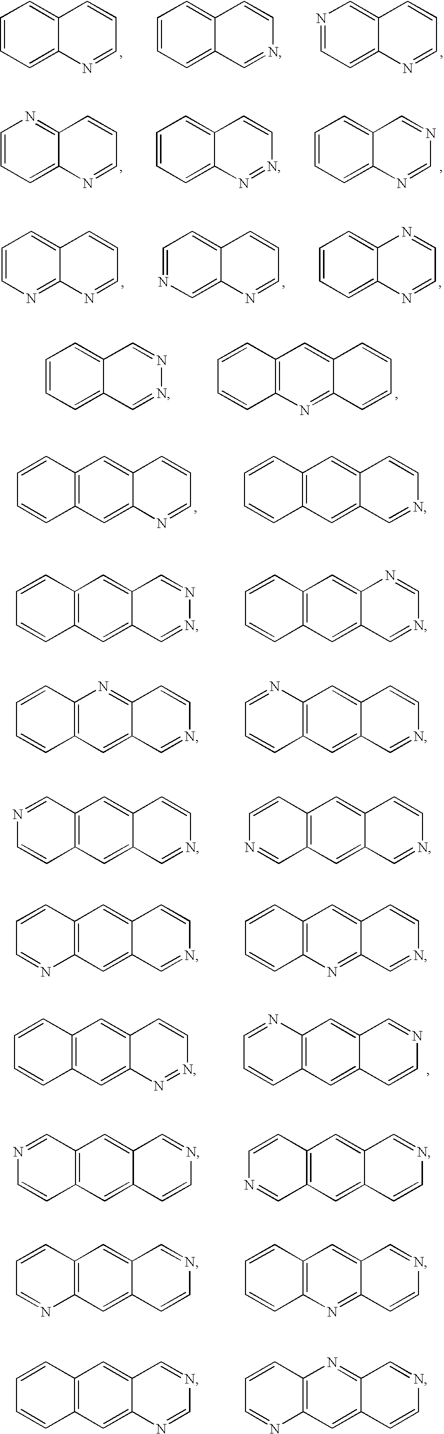

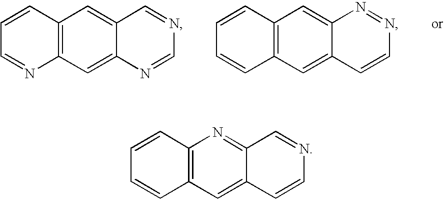

14. The compound of claim 10 further comprising a pharmaceutical composition comprising a pharmaceutically acceptable carrier, excipient, or diluent.

15. A compound of the formula: 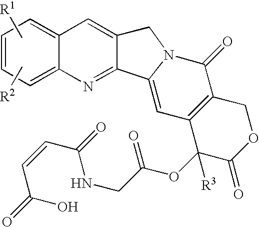
or a pharmaceutically acceptable salt, entantiomer or disastereomer thereof, wherein
R′ and R² are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—(C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C₁-C₈)alkynyl, or S-aryl; O—S(O)₂—(C₁-C₈)alkyl, O—S(O)₂-(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer having a value of 1 to 7; and
R³ is H, (C₁-C₈)alkyl, (C₁-C₈)alkenyl, or (C₁-C₈)alkynyl.

16. The compound of claim 15 wherein:
R¹ is H or a group of the formula: 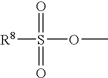
wherein R⁸ is (C₁-C₈)alkyl, (C₁-C₈)alkenyl, (C₁-C₈)alkynyl, or aryl;
R² is H or a group of the formula: 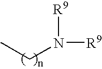
wherein each occurrence of R⁹ is independently H, (C₁-C₈)alkyl, (C₁-C₈)alkenyl, (C₁-C₈)alkynyl, or aryl, and n is an integer having a value of 1-7;

17. The compound of claim 16 wherein R³ and R⁸, and R⁹ are independent (C₁-C₈)alkyl groups.

18. The compound of claim 15 further comprising a pharmaceutical composition comprising a pharmaceutically acceptable carrier, excipient, or diluent.

19. A compound of the formula: 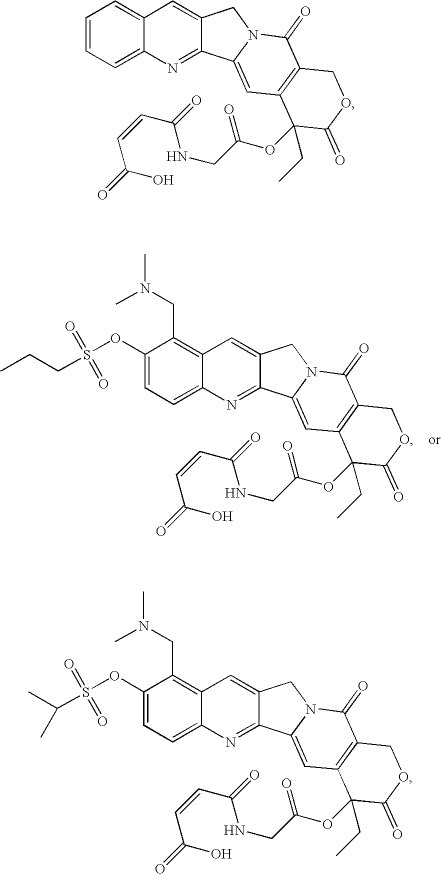
or a pharmaceutically acceptable salt, entantiomer or disastereomer thereof.

20. A method of treating cancer in a mammal comprising administering to a mammal in need of said treatment a therapeutically effective amount of a compound of the formula: 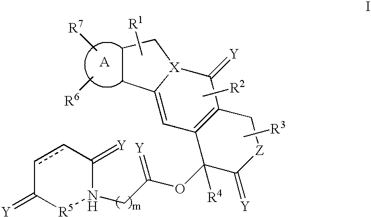
or a pharmaceutically acceptable salt, entantiomer or disastereomer thereof, wherein the “A” ring is a nitrogen-aryl group;
each occurrence of Y is independently oxygen or H₂;
Z is oxygen or —CH₂—;
X is nitrogen or —CH;
R¹, R², R³, R⁴, R⁵, R⁶, and R⁷ are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—(C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C₁-C₈)alkynyl, S-aryl; O—S(O)₂—(C₁-C₈)alkyl, O—S(O)₂—(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer from 1-7;
m is an integer having a value of 1-5; and
the dotted line represents an optional bond.

21. The method of claim 20 wherein:
R⁶ is H or a group of the formula: 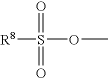
wherein R⁸ is (C₁-C₈)alkyl, (C₁-C₈)alkenyl, (C₁-C₈)alkynyl, or aryl; and
R⁷ is H or a group of the formula: 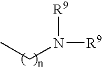
wherein each occurrence of R⁹ is independently H, (C₁-C₈)alkyl, (C₁-C₈)alkenyl, (C₁-C₈)alkynyl, or aryl, and n is an integer having a value of 0-7.

22. A method of treating cancer in a mammal which comprises administering to a mammal in need of said treatment a therapeutically effective amount of a the formula: 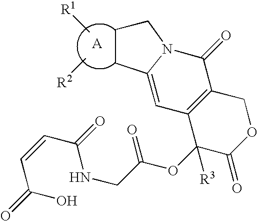
or a pharmaceutically acceptable salt, entantiomer or disastereomer thereof, wherein, the “A” ring is a nitrogen-aryl group;
R¹ and R² are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—(C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C_{C}₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C₁-C₈)alkynyl, S-aryl; O—S(O)₂—(C₁-C₈)alkyl, O—S(O)₂—(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer from 1-7; and
R³ is H, (C₁-C₈)alkyl, (C₁-C₈)alkenyl, or (C₁-C₈)alkynyl.

23. The method of claim 22 wherein R′ is H or a group of the formula: 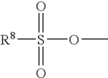
wherein R⁸ is (C₁-C₈)alkyl, (C₁-C₈)alkenyl, (C₁-C₈)alkynyl, or aryl; and
R² is H or a group of the formula: 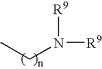
wherein each occurrence of R⁹ is independently H, (C₁-C₈)alkyl, (C₁-C₈)alkenyl, (C₁-C₈)alkynyl, or aryl, and n is an integer having a value of 0-7.

24. A method of treating cancer in a mammal which comprises administering to a mammal in need of said treatment a therapeutically effective amount of a compound of the formula: 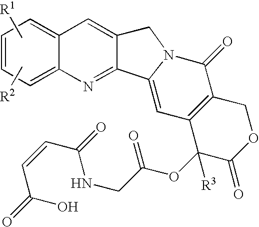
or a pharmaceutically acceptable salt, entantiomer or disastereomer thereof, wherein
R¹ and R² are independently H, (C₁-C₈)alkyl, (C₁-C₈)alkenyl, (C₁-C₈)alkynyl, aryl, (C₂-C₅)heteroaryl, (C₁-C₆)heterocycloalkyl, (C₃-C₇)cycloalkyl, O—(C₁-C₈)alkyl, O—(C₁-C₈)alkenyl, O—(C₁-C₈)alkynyl, O-aryl, CN, OH, oxo, halo, C(O)OH, C(O)halo, O(CO)halo, CF₃, N₃, NO₂, NH₂, NH((C₁-C₈)alkyl), N((C₁-C₈)alkyl)₂, NH(aryl), N(aryl)₂, (CO)NH₂, (CO)NH((C₁-C₈)alkyl), (CO)N((C₁-C₈)alkyl)₂, (CO)NH(aryl), (CO)N(aryl)₂, O(CO)NH₂, NHOH, NOH((C₁-C₈)alkyl), NOH(aryl), O(CO)NH((C₁-C₈)alkyl), O(CO)N((C₁-C₈)alkyl)₂, O(CO)NH(aryl), O(CO)N(aryl)₂, CHO, CO((C₁-C₈)alkyl), CO(aryl), C(O)O((C₁-C₈)alkyl), C(O)O(aryl), O(CO)((C₁-C₈)alkyl), O(CO)(aryl), O(CO)O((C₁-C₈)alkyl), O(CO)O(aryl), S—(C₁-C₈)alkyl, S—(C₁-C₈)alkenyl, S—(C_{-C}₈)alkynyl, or S-aryl; O—S(O)₂—(C₁-C₈)alkyl, O—S(O)₂—(C₁-C₈)alkenyl, O—S(O)₂—(C₁-C₈)alkynyl, O—S(O)₂-aryl, (CH₂)ₙ—NH₂, (CH₂)ₙ—NH((C₁-C₈)alkyl), (CH₂)ₙ—N((C₁-C₈)alkyl)₂, (CH₂)ₙ—NH(aryl), or (CH₂)ₙ—N(aryl)₂;
n is an integer having a value of 1 to 7; and
R³ is H, (C₁-C₈)alkyl, (C₁-C₈)alkenyl, or (C₁-C₈)alkynyl.

25. The method of claim 24 wherein:
R¹ is H or a group of the formula: 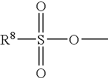
wherein R⁸ is (C₁-C₈)alkyl, (C₁-C₈)alkenyl, (C₁-C₈)alkynyl, or aryl;
R² is H or a group of the formula: 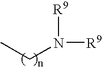
wherein each occurrence of R⁹ is independently H, (C₁-C₈)alkyl, (C₁-C₈)alkenyl, (C₁-C₈)alkynyl, or aryl, and n is an integer having a value of 1-7.

26. A method of treating cancer in a mammal which comprises administering to a mammal in need of said treatment a therapeutically effective amount of a compound of the formula: 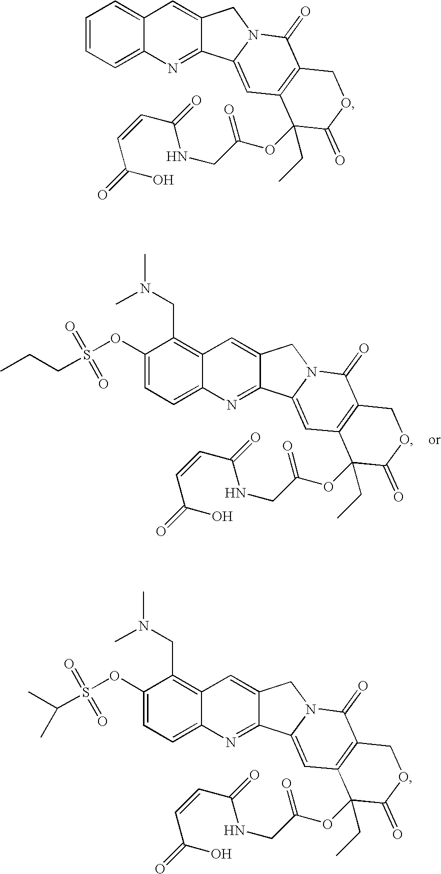
or a pharmaceutically acceptable salt, entantiomer or disastereomer thereof.
